(19) United States
(12) Patent Application Publication
(10) Pub. No.: US 2011/0092678 A1
(43) Pub. Date: Apr. 21, 2011

(54) SINGLE-LABELLING AGENTS BASED ON VINYL SULPHONE

(76) Inventors: Francisco Santoyo González, Granada (ES); Fernando Hernández Mateo, Granada (ES); Francisco Javier Lopez Jaramillo, Granada (ES); Mariano Ortega Muñoz, Granada (ES)

(21) Appl. No.: 12/920,033

(22) PCT Filed: Feb. 19, 2009
(86) PCT No.: PCT/ES09/70034

§ 371 Date: Dec. 3, 2010

(30) Foreign Application Priority Data
Feb. 19, 2008 (ES) P200800576

(51) Int. Cl. C07D 495/04; C07C 381/00; C07D 311/80; C07K 2/00

(52) U.S. Cl. 530/408; 564/500; 548/304.1; 549/394

(57) ABSTRACT

The invention relates to labelling agents containing a compound with a labelled molecule and a vinyl sulphone group. The invention also relates to the compounds, the method for obtaining them and the uses thereof in the marking of biomolecules and, more specifically, proteins.

The present invention refers to a compound of the general formula (I) comprising a labelled molecule and vinyl sulphone groups, which function is to make the covalent binding with the molecules susceptible to be labelled, it also refers to the procedures for obtaining them and their uses. More particularly, it refers to the use of these compounds for the labelling of biomolecules and their biotechnological applications.

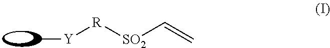

### PRIOR STATE OF THE ART

The labelling of biomolecules is a basic tool in the field of genomics and proteomics for the detection, purification and study of interactions between biomolecules.

From the range of biomolecule labellings which are plausible, there stand out by their special importance the labelling with fluorophores and biotin due to their biotechnological applications and their commercial impact.

Fluorescent labelling is a key element for the detection and analysis of biomolecules (Patton, W. F. Electrophoresis (2000), vol. 21, pp. 1123-1144) and it is the engine of a multi-million euro industry. The advantages of fluorescent labelling vis-á-vis conventional methods such as Coomassie blue (Wang, X. et al. Biotechnol. Lett. (2007), vol. 29, pp. 1599-1063), silver (Rabillould, T. Electrophoresis (1990), vol. 11 pp. 785-794), colloidal gold (Rohringer, R.; Holden, D. W., Anal. Biochem. (1985), vol. 144, pp. 118-127) or radioactivity (Waggoner, A., Curr. Opin. Chem. Biol. (2006), vol. 10, pp. 62-66), are the following:
Rapid and high sensitivity detection: each fluorescent label can originate 10⁷-10⁸ photons per second.
Versatility: Different labellings originate different “colours”, being possible to make a “polychromatic” labelling such as that used, for example, in DNA sequentiation (Smith, L., et al., Nature (1986), vol. 321, pp. 674-679).
Inertia: Fluorophore size and properties rarely intervene with the marked molecules.
Localization of the signal in the labelling point, unlike enzymatic labelling.

However, its potential goes beyond passive detection since techniques such as fluorescence polarization and FRET (Fluorescence Resonance Energy Transfer, also called Förster Resonance Energy Transfer) enable to evaluate conformational changes, interactions between proteins and between protein and ligand. The measurement of the polarization provides information on orientations and mobility which enables to study receptor-ligand interactions (Jameson, D. M., Seifried, S. E., Methods (1999), vol. 19, pp. 222-233), and FRET is an interaction between fluorophores in which the excitation passes from an excited fluorophore (donor) to another which is excited (acceptor) without the emission of a photon. This interaction is produced when the donor emission wavelength is very close to that of the acceptor excitation and is very dependent on the distance between the donor and the acceptor, so it was used as rule (Remedios, C. G., Moens, P. D., J. Struct. Biol. (1995), vol. 115, pp. 175-185) to analyse conformational changes and interaction between biomolecules.

Nowadays, there exists a great amount and variety of fluorophores. Among the ones used for biomolecule labelling, we can mention dansyl, fluorescein and rhodamine B, whose functional characteristics and some of their applications are summarized in the table below:

| | Λ | Λ | |
|---|---|---|---|
| | absorp- | emis- | |
| Fluorophores | tion | sion | Some applications |
| Dansyl | 335 nm | 518 nm | Labelling for detection in general |
| | | | Quantum yield depending on the |
| | | | medium: receptor-ligand interaction |
| | | | analysis FRET with Tryptophan |
| | | | (donor) and with fluorescein |
| | | | (acceptor) (Gettins, P. G. W., Olson, |
| | | | S. T. Methods (2004), vol. 32, |
| | | | pp. 110-119) |
| Fluorescein | 494 nm | 518 nm | Labelling for detection in general |
| | | | Application in fluorescence polar- |
| | | | ization FRET with Rhodamine |
| | | | (acceptor) (Ghosh, S. S., et al., |
| | | | Nucleic Acids Res. (1994), vol. 22, |
| | | | pp. 3155-3159) |
| | | | Homo-FRET (Hamman, B. D., et al., |
| | | | Biochemistry (1996), vol. 35, pp. |
| | | | 16680-16686) |
| Rhodamine | 543 nm | 565 nm | Labelling for detection in general |
| B | | | Application in fluorescence polar- |
| | | | ization FRET with fluorescein or |
| | | | dansyl (donors) (Yegneswaran, S., |
| | | | et al., J. Mol. Biol. (2003), vol. |
| | | | 278, pp. 14614-14621) |

On the other hand, labelling with biotin is also very important in biotechnology (Wilchek, M.; Bayer, E. A., Anal. Biochem. (1988), vol. 171, pp. 1-32). Biotin is a molecule which acts as coenzyme of certain carboxylases related to the metabolism of carbon dioxide. However, its biotechnological interest lies in the high specificity and affinity which avidin, streptavidin and other related proteins have for this biomolecule (dissociation constant around 10⁻¹⁵-M⁻¹), causing the interaction to have the strength of a covalent bond without being one. Thus, the biotinylation transforms molecules which are hard to detect in probes which can be detected or captured with marked or immobilized avidin/streptavidin. This principle is common to find antigens in tissues, cells and to detect biomolecules in immunoassays and in DNA hybridization tests. However, for certain applications, such as for example purification through affinity chromatography, it is necessary that the biotin-avidin interaction is reversible, for which adivin can be modified (by nitrosylation of tyrosines of the active centre (Morag, E., et al., Biochem. J., (1996), vol. 316: pp. 193-199)) or biotin derivatives can be used (desthiobiotin and iminobiotin). There exist biotins fluorescently marked to quantify active sites of avidin (Gruber, H. J, et al., Biochim. Biophvs. Acta (1998), vol. 1381, pp. 203-212) and biotin labelled with DNP (DNP-X-biocytin-X; U.S. Pat. No. 5,180,828A) (dinitrophenol), versatile labelling which besides acting as chromophore is recognized by antibodies anti-DNP, allowing the correlation between fluorescence and electronic microscopy studies. There also exists in the market Horseradish peroxidase (HRP) labelled with biotin.

A fundamental aspect vis-á-vis the use of any labelling is the binding to the biomolecule and the stability of said binding. From a chemical point of view there exist four groups present in the biomolecules susceptible of acting as targets for the anchorage of the labelling reagents conveniently derivatized through the formation of a covalent bond, such as amines, thiols, alcohols and carboxylic acids, which are detailed below:

Amines: They are the most common target of reagents of covalent modification and the main one in proteins. In most of these biomolecules the amino end is free and almost all have lysine, residue in whose side chain there is a ε-amino group easily modifiable since it is mostly found in the surface of proteins. These groups react with acylating reagents and the reactivity depends on the acylating reactive, the type of amine, basicity and pH of the reaction. Aliphatic amines, such as that of the side chain of lysine, are moderately basic and react with most acylating reagents to pH higher than 8.

There are three derivatizations of labelling reagents which react with amines of biomolecules:
Succinimidyl esters: They react with amines to originate amides. It is the most frequent derivatization given the stability of the amide bond which is generated. They react well with aliphatic amines and have low reactivity with aromatic amines, alcohols, phenols (tyrosine) and imidazole. In presence of thiols (cysteine) they can form thioesters but in proteins the acyl group can be transferred to a neighbour amine. One of the main inconveniences of succinimidyl esters is their solubility, which in some cases can be very low. Therefore, in the market there exist carboxylic acid derivatives which can be converted into succinimidyl esters (Staros, J. V., et al., Anal. Biochem. (1986), vol. 156, pp. 220-222) or STP esters (Gee, K. R., et al. Tetrahedron Lett. (1999), vol. 40, pp. 1471-1474), which are more polar, and therefore more water-soluble, although less reactive with amines with little exposure.
Isothiocyanates: They react with amines to form thioureas, which are reasonably stable in most cases.
Sulfonic acid chlorides: They react with amines and produce sulfonamides. They are very reactive and unstable in aqueous means, especially to alkaline pH necessary for them to react with aliphatic amines, so work is done at low temperature. Once conjugated, the bond is extremely stable and resistant. They also react with phenols (tyrosine), aliphatic alcohols (polysaccharides), thiols (cysteine) and imidazoles (histidine) although those conjugated with thiols and imidazoles are unstable and those conjugated with aliphatic alcohols can undergo nucleophilic displacements.
Other functionalizations can be: aldehydes and arylating agents. Aldehydes which react with amines to form Schiff bases. There have been prepared o-phthalaldehyde (OPA), naphthalene dicarboxaldehyde (NDA) and 3-acryl quinoline carboxaldehyde (OTTO-TAG) and there have been used for quantification of amines in solution (Liu, J., Hsieh, et al., Anal. Chem. (1991), vol. 163, pp. 408-412). And arylating agents such as 4-nitro-2,1,3-benzoxadiazol (NBD) chloride or fluoride (Watanable, Y., Imai, K., J. Chromatogr. (1982), vol. 239, pp. 723-732).

Thiols: They are more selective targets than the amine group, as they are less frequent in biomolecules and to be reagents they have to be free (but not form a disulphide brigde). The sulfhydryl group can be introduced in the macromolecule to mark through chemical modification, reduction of disulphide brigdes or intein path (Tan, L. P., Yao, S. Q. Protein and Pept. Lett. (2005), vol. 12, pp. 769-751) (in the case of proteins), or through directed mutagenesis to introduce cysteine.

Thiol groups react to physiological pH (pH 6, 5-8) with alkylating reagents (such as iodoacetamides and maleimides) or arylating reagents (such as 7-choro or 7-fluoro-4-nitro-2,1,3-benzoxadiazol (NBD)), to originate stable thioethers. They also react with many of the acylating reagents of amines, including isothiocyanates and succinimidyl esters. Symmetric disulphides such as didansyl-L-cysteine or 5,5′-dithiobis-(2-nitrobenzoic) acid (DTNB) (Daly, T J., et al., Biochemistry (1986), vol. 25, pp. 5468-5474) also react with the thiols to give bindings of the non-symmetric disulphide type.

Alcohols: The hydroxyl function is present in the side chains of tyrosine, serine and threonine, in sterols and carbohydrates, but its reactivity in aqueous solutions is extremely low, especially in proteins due to the presence of more active nucleophiles such as amines and thiols. A function which reacts specifically with neighbour diols is boronic acid and forms cyclical complexes (Gallop, P. M., et al., Science (1982), vol. 217, pp. 166-169). However, a standard procedure to increase reactivity, especially in the case of carbohydrates, is oxidation with periodate to give origin to the aldehyde function. The main functionalizations of labelling reagents which react with the aldehyde function of biomolecules are: amine, hydrazides, semicarbazide, carbohydrazide and O-alkylhydroxylamines.

Carboxylic acid group: They are abundant in macromolecules but little reactive, so their derivatization is usual so that amines are inserted which react with the functionalizations described above.

Nowadays, it is possible to commercially acquire an entire range of labelling products with conveniently derivatized fluorescence and with biotin. The most frequent strategy to functionalize labelling reagents is the derivatization as succinimidyl esters to react with the amine functions of the biomolecule.

On the other hand, and from a chemical perspective, α,β-unsaturated sulphones (vinyl sulphones) are known as synthetic intermediaries greatly useful mainly because of their capacity to participate in 1,4-addition reactions (Michael acceptors). Additionally, vinyl sulphones are easy to prepare, through a wide variety of synthetic processes, and to manipulate (Simphinks, N. S., Tetrahedron (1990), vol. 282, pp. 6951-6984). These characteristics have recently been found useful in the design of drugs and in medicinal chemistry when their capacity to powerfully and reversibly inhibit a variety of enzymatic processes, mainly those involving cysteine proteases to which they are joined through addition reactions with the thiol group present in the cysteine residue of the active site of these enzymes, was proved (Meadows, D. C., et al. Med. Res. Rev. (2006), vol. 26(6), pp. 793-814).

However, from a biotechnological viewpoint, their potential goes beyond that. The reactivity of vinyl sulphones with biomolecules has been harnessed for the introduction of polyethylene glycol through reaction with thiols (Morpurgo, M., et al., Bioconiug. Chem. (1996) vol. 7, pp. 363-368), for the formation of hydrogels through peptide crosslinking with polyethylene glycol functionalized with vinyl sulphone (Rizzi, S. C, et al., Biomacromolecules (2006), vol. 7, pp. 3019-3029) and for the introduction of derivatized glucose molecules with vinyl sulphone through reaction with the amines of the proteins (López-Jaramillo, et al., Acta Cryst. (2005) vol. F61, pp. 435-438).

As markers, there have been described coloured compounds containing vinyl sulphone groups. In this sense, U.S. Pat. No. 4,473,693 describes coloring agents, for intracellular marking, based on Lucifer yellow and containing a vinyl sulphone group. In patent EP0187076 there are described fluorescent compounds containing a vinyl sulphone group, these compounds are useful for immunologic studies.

### EXPLANATION OF THE INVENTION

In the present invention it is provided a new compound of general formula (I) which comprises a labelling molecule, as well as a vinyl sulphone group, and which allows carrying out the labelling of biomolecules in a highly effective and simple manner. These compounds constitute an alternative to the derivatization currently used in proteomics and genomics for introducing a labelling reactive into biomolecules.

Therefore, a first aspect of the present invention refers to the compounds of general formula (I) (hereinafter compounds of the invention):

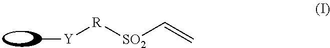

where:

Y is a radical selected among an oxygen atom (O) or from the group, substituted or non-substituted, —N(R¹)CH₂CH₂SO₂, where: R¹ is a radical, substituted or non-substituted, that is selected among the group comprising an alkyl (C₁-C₁₀) or from the group (CH₂)ₘC≡CH; where m is a value from 1 to 10, preferably m is a value from 1 to 5, more preferably m is 1, 2, or 3, even more preferably m is 1. When R¹ is an alkyl group, it is preferably an alkyl (C₂-C₆), more preferably an alkyl C₄ and even more preferably a sec-butyl group.

R is a radical, substituted or non-substituted, which is selected from the group comprising the following formulas. —R²OCH₂CH₂, —CH₂CH₂OR²OCH₂CH₂, or —(CH₂CH₂O)ₙCH₂CH₂, where R² is an alkyl radial (C₁-C₁₀), substituted or non-substituted, or a dialkylaryl radical ((C₁-C₁₀)Ar(C₁-C₁₀)), substituted or non-substituted; and n is a value from 2 to 20; preferably R is a group of formula —(CH₂CH₂O)ₙCH₂CH₂, n may be a value from 2 to 10, more preferably n is 2, 3, 4, 5 or 6; even more preferably n may be 2 or 4; and

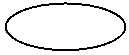

represents a labelling molecule.

The term “alkyl” in the present invention refers to aliphatic chains, lineal or branched, that have from 1 to 10 carbon atoms, for example, methyl, ethyl, n-propyl, i-propyl, n-butyl, tert-butyl, sec-butyl, n-pentyl, etc. Preferably the alkyl group has from 2 to 6 carbon atoms.

The term “dialkylaryl” in the present invention refers to an aryl group which is substituted with two alkyl groups that have from 1 to 10 carbon atoms, more preferably have from 1 to 5 carbon atoms. The alkyl groups may be equal or different, preferably they are equal and the term “aryl” in the present invention refers to an aromatic carbocyclic chain, which has from 6 to 12 carbon atoms, may have a single or multiple rings, separated and/or condensed. The typical aryl groups contain from 1 to 3 separated or condensed rings and from 6 to approximately 18 ring carbon atoms, such as phenyl, naphthyl, indenyl, phenantryl or antracyl radicals.

The term “labelling molecule” refers in this description to any biorecognizable substance, colorant, fluorophore or any other group detectable by techniques for spectrophotometry, fluorometry, optical microscopy, fluorescence or confocal microscopy, antibodies and/or NMR, and which easily allows the detection of another molecule that is not easily detected and/or quantified on its own. Preferably, this labelling molecule is biotin or a fluorophore selected from among fluorescent markers containing at least a carboxylic acid group or a sulphonic acid group, henceforth represented according the figures:

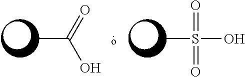

More preferably, the fluorophore may be dansyl, rhodamine or any derivative thereof.

Derivatives of the labelling molecules may be acid or sulphonyl halogenides, and more preferably acid or sulphonyl chlorides.

A second aspect of the present invention relates to a method for obtaining the compounds of the invention, that is, the compounds of general formula (I), and which comprises:

reacting the functionalized vinyl sulphones of general formula (II), which present, a vinyl sulphone group, as well as one or two additional functional groups for the binding with the labelling molecules:

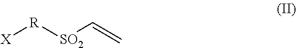

where: R is previously defined; and
X is —OH or the group —SO₂CH₂CH2₂NH(R¹); where R¹ is previously defined.
   with a labelling molecule that contains a carboxylic acid or sulphonic acid which allows through them or one of the activated derivatives thereof the formation of:
an amide or sulfonamide bond with the vinyl sulphones of general formula (II) when X represents the group —SO₂CH₂CH2₂NH(R¹); or
an ester or sulfonate bond when X represents an —OH group; according to the following scheme:

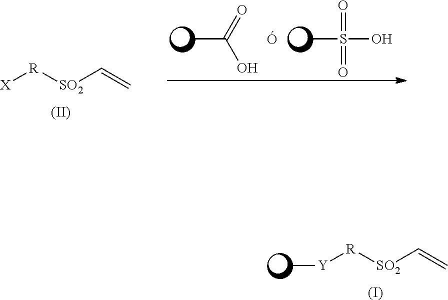

In a preferred embodiment of the present invention, acid chlorides or sulphonyl chlorides derivatives of the labelling molecules are used and the compounds of the invention are obtained by reaction of these derivatives with the vinyl sulphones of general formula (II) through:

a) esterification reactions with acid chlorides of the labels when X is —OH; b) amidation reactions with acid chlorides or sulphonyl chlorides of the labels when X is —SO₂CH₂CH2₂NH(R¹). In this way it can be obtained biotinylation labelling compounds and fluorescent labelling compounds containing dansyl or rhodamine as fluorophores.

A preferred embodiment of the method of the present invention comprises functionalized vinyl sulphones of general formula (II) where X is —OH, —SO₂CH₂CH2₂NHCH(CH₃)CH2₂CH₃ or —SO₂CH₂CH2₂NHCH₂C≡CH; R is (CH₂CH2₂O)ₙCH₂CH2₂ and n may take values between 2 and 4. That is, preferred vinyl sulphones of general formula (II) are the following:

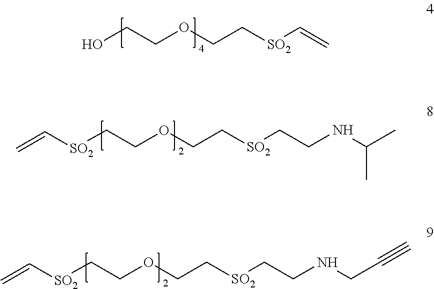

In another preferred embodiment these compounds of general formula (II) are obtained by reaction of divinyl sulphone (DVS) with diols (formula (III)):a) in a 1:1 ratio to provide the ω-hydroxy vinyl sulphone, when X is —OH (corresponds to the compound of general formula (IV)); or b) in ≧2:1 ratio to provide bis-vinyl sulphones, corresponding to the compound of general formula (V), which are subsequently transformed by reaction of one of the vinyl sulphone groups with primary amines through a Michael-type addition reaction providing the corresponding amine vinyl sulphone, that is, the compounds of general formula (II) when X is —SO₂CH₂CH₂NH(R¹), which corresponds, in the following scheme, to the compound of general formula (VI).

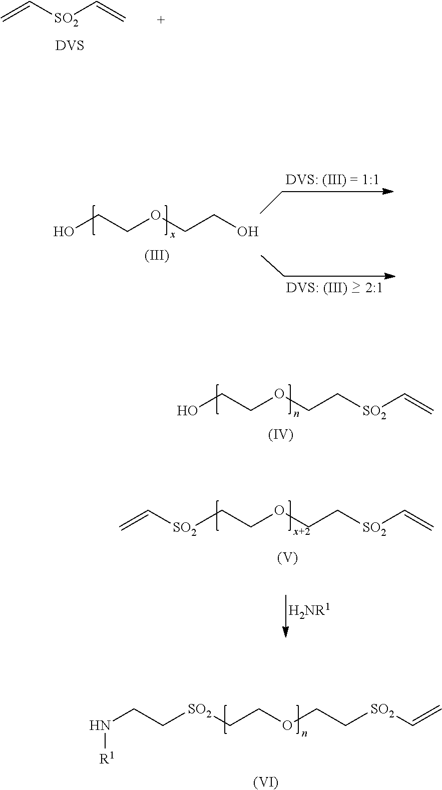

where: R¹ and n are previously defined.
   x takes values from 0 to 19 and, n is related to x in the following way: n is x+1 in the general formula (IV) and n is x+2 in the general formula (VI).

In an even more preferred embodiment, these diols are tetraethylene glycol (when x is 3) and ethylene glycol (when x is 0).

In this way, it can be provided difunctional (compounds 4 and 8) and tri-functional (compound 9) compounds with groups that present an orthogonal reactivity to each other, circumstance that allows modulating their reactivity. Thus, according to the method of the present invention the vinyl sulphones of general formula (II) allow carrying out the incorporation of any labelling molecule that contains functional groups with reactivity complementary to the groups present in them and that leave unchanged a vinyl sulphone group which is used for the subsequent binding to biomolecules. In particular, and since vinyl sulphones of formulas (II) of the preferred embodiment of the present invention are carriers of the hydroxyl and amino functions, it can be used, but without limiting to, labelling molecules derivatives containing a) the acid chloride or b) sulphonyl chloride function.

In this way, the derivatives of these preferred labelling molecules may be the following:

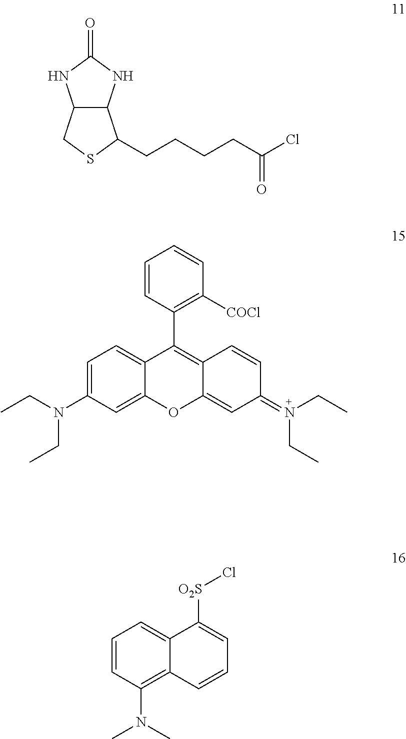

The compound of the invention provides a labelling technique which is based on the chemoselective ligation of the vinyl sulphone function with complementary groups present in a natural way in any biomolecule (amino groups or thiol groups) and with which it reacts through Michael-type addition reactions. Besides, the compound is compatible with the biological nature of biomolecules and the technique does not require any activation strategy.

The use of the vinyl sulphone function as derivatization of the labelling reagents for carrying out the covalent binding biomolecule-compound of the invention presents the following advantages:

a) Stability of the labelling agents containing such function.
b) Formation of a stable covalent binding.
c) Fast reaction with high yields without the generation of any type of by-product.
d) No large excesses of reagents required.
e) The reactions are carried out in the absence of catalysts by simple mixture of the reagents.
f) The reactions may be carried out in water without the use of co-solvents.
g) The reactions may be carried out under physiological conditions:
aqueous medium, narrow pH range, mild temperatures.
h) Simple purification and isolation processes.
i) There exists a tolerance towards the other functional groups present in biomolecules different from the amino and thiol groups with which the vinyl-sulphones react.

Therefore, another aspect of the present invention refers to the use of the compounds of general formula (I) as labelling agents for the marking or labelling of molecules, and more preferably of biomolecules. In the present invention the term “labelling agent” refers to those compounds which are able of binding to a molecule and which also allow displaying, detecting and/or quantifying by means of spectroscopy (absorption, fluorescence, NMR and others), enzymatic reactions (peroxidase, alkaline phosphatase and others) or spectrometry (mass and others) of the molecule object of the marking.

In a preferred embodiment of the present invention, biomolecules are proteins.

In an even more preferred embodiment of the present invention, the proteins are selected from the group comprising Bovine Serum Albumin (BSA), lysozyme, GFP (Green Fluorescent Protein), Concanavalin A, Avidin or crude pea extract.

In a preferred embodiment of the present invention protein labelling is carried out in a solution without free amines such as, but without limiting to, phosphate or HEPES, at moderate ionic strength, (50-200 mM) and basic pH (7.5-8.7) and the reaction with an excess of the labelling reagents of general formula (I) during an appropriate time (usually all night long at room temperature) being the reagent excess eliminated by dialysis (Scheme 1).

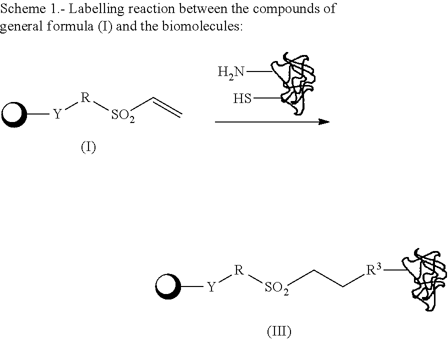

where:
Y and R are previously defined;

### R³ is NH or S; and

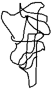

represents the biomolecule

Throughout the description and the claims the word “comprise” and its variants are not intended to exclude other technical features, additives, components or steps. For the subject experts, other objects, advantages and features of the invention will be inferred in part from the description and in part from the practice of the invention. The following examples and figures are provided as an illustration, and are not intended to be limitative to the present invention.

### DESCRIPTION OF THE DRAWINGS

FIG. 1. Shows the avidin marking with compound 18, originating fluorescence (gel on the left (A)) and compatible with the subsequent Coomassie staining (gel on the right (B)). The samples are, from left to right:

row 1: stoichiometry 1:4/3 hours
row 2: stoichiometry 1:4/8 hours
row 3: stoichiometry 1:4/24 hours
row 1: stoichiometry 1:8/3 hours
row 2: stoichiometry 1:8/8 hours
row 3: stoichiometry 1:8/24 hours

FIG. 2. Shows the concanavalin A marking with compound 17, originating fluorescence (gel on the left (A)) and compatible with Coomassie (gel on the right (B)). The samples are, from left to right:

row 1: stoichiometry 1:5/3 hours
row 2: stoichiometry 1:5/8 hours
row 3: stoichiometry 1:5/24 hours
row 1: stoichiometry 1:10/3 hours
row 2: stoichiometry 1:10/8 hours
row 3: stoichiometry 1:10/24 hours

FIG. 3. Shows the labelling previous to electrophoresis of BSA and lysozyme with compound 17, originating fluorescence (gel on the left (A)) that allows detecting “de visu” by the order of 125 ng and is compatible with a subsequent silver staining after the electrophoresis (gel on the right (B)).

The samples are, from left to right:

### row 1: BSA-rhodamine

row 2: lysozyme-rhodamine
row 3: non-labelled BSA (control)
row 4: non-labelled lysozyme (control)

FIG. 4. Shows the labelling previous to electrophoresis of crude pea extract with compound 17, allows its analysis with no need of a subsequent Coomassie or silver staining.

FIG. 5. Shows the detection of BSA marked with biotin (stoichiometries BSA:biotin in brackets) with different stoichiometries of fluorescent avidin. From left to right:

row 1: Avidin-Dansyl: BSA-biotin (1:10) stoichiometry 1:1
row 2: Avidin-Dansyl: BSA-biotin (1:10) stoichiometry 4:1
row 3: Avidin-Dansyl: BSA-biotin (1:5) stoichiometry 1:1
row 4: Avidin-Dansyl: BSA-biotin (1:5) stoichiometry 4:1
row 5: Control of Avidin-Dansyl with BSA-biotin

### EXAMPLES

There follows an illustration of the invention by means of some assays carried out by the inventors, which prove the specificity and effectiveness of the compounds of the invention.

### Example 1

### Synthesis of Vinyl Sulphones of Formula (II): Compounds 4, 8 and 9

The vinyl sulphones of general formula (II) were obtained from divinyl sulphone (DVS) and diols, (a) in a 1:1.2 ratio to provide the ω-hydroxy vinyl sulphone (compound 4) or (b) in a 3:1 ratio to provide bisvinyl sulphones (compounds 5) that are subsequently transformed by reaction with primary amines from one of the vinyl sulphone groups through a Michael-type addition reaction providing the corresponding amino vinyl sulphone (compound 8 and 9).

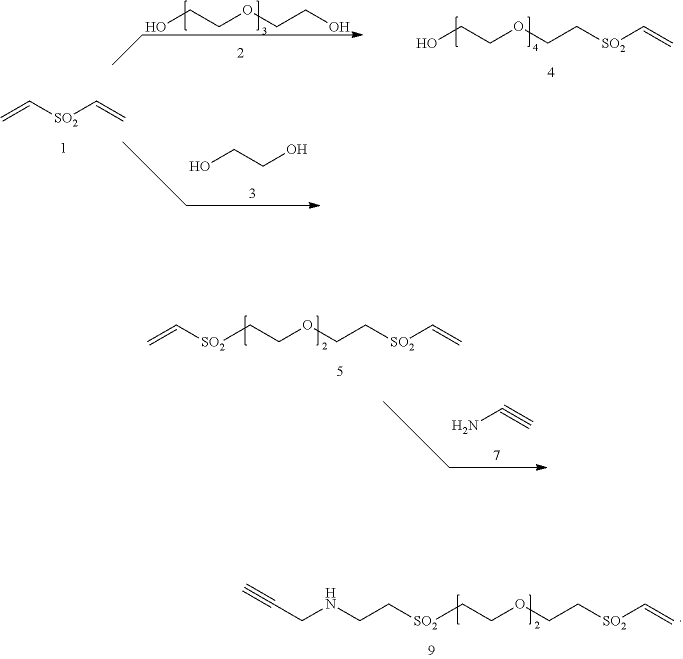

Compound 4: DVS 1 (1.1 mL, 11 mmol) and DBU (690 mg, 4.5 mmol) was added to a solution of tetraethylene glycol 2 (1.760 g, 9.07 mmol) in CH₂Cl₂ (20 mL). The reaction mixture was left at room temperature (16 h.). The solvent was eliminated by vacuum evaporation. The crude obtained was purified by column chromatography (AcOEt-MeOH 10:1) obtaining 4 as a liquid (1.07 g, 38%).

Compound 5: DVS 1 (1.6 mL, 16 mmol) and t-BuOK (119 mg, 1.1 mmol) was added to a solution of ethylene glycol 3 (330 mg, 5.3 mmol). The reaction mixture was left at room temperature (30 min.). The solvent was eliminated by vacuum evaporation. The crude obtained was purified by column chromatography (AcOEt-hexane 2:1 to 3:1) obtaining 5 as a syrup (800 mg, 51%).

Compound 8: Sec-butylamine 6 (164 mg, 2.2 mmol) was added to a solution of 5 (1.0 g, 3.3 mmol) in CH₂Cl₂-isopropanol 2:1. The reaction mixture is left at room temperature (6 h.). The solvent was eliminated by vacuum evaporation obtaining a crude that was purified by column chromatography (AcOEt to AcOEt-MeOH 10:1) obtaining 8 as a syrup (472 mg, 57%).

Compound 9: Propargylamine 7 (51 mg, 0.93 mmol) was added to a solution of 5 (414 mg, 1.4 mmol) in CH₂Cl₂-isopropanol 2:1. The reaction mixture was left at room temperature (1 day). The solvent was eliminated by vacuum evaporation obtaining a crude that was purified by column chromatography (AcOEt to AcOEt-MeOH 10:1) obtaining 9 as a syrup (170 mg, 52%).

### Example 2

### Synthesis of Single-Labelling Agents Based on Vinyl Sulphone Containing Biotin: Compounds 12-14

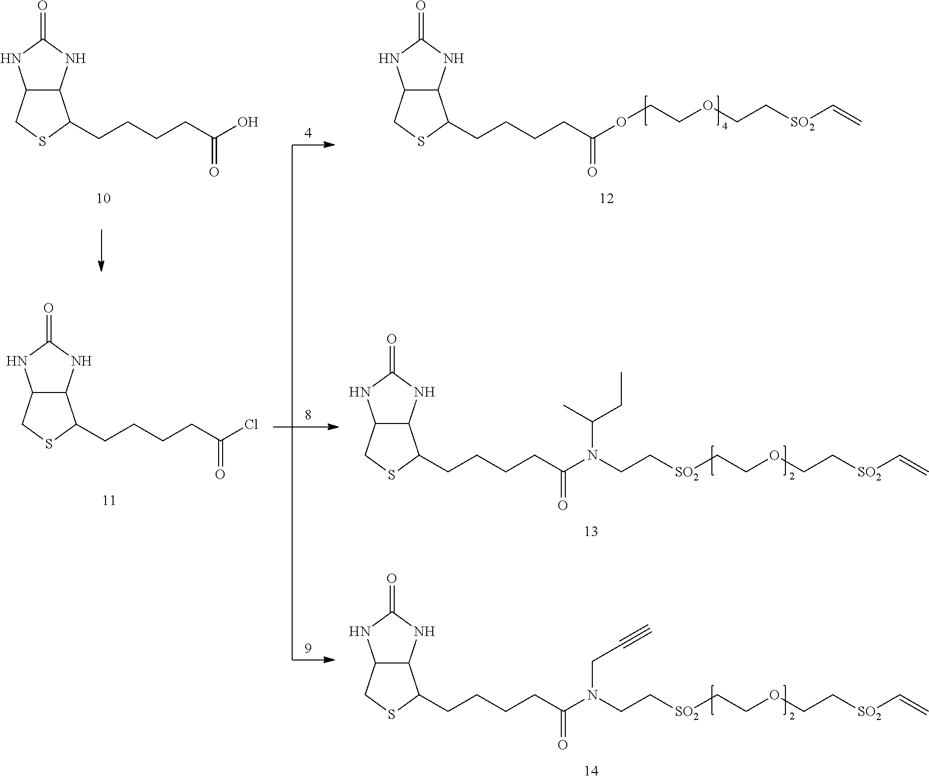

Compound 12: A solution of biotin 10 (247 mg, 1 mmol) in Cl₂SO (5 mL) was kept at room temperature (1 h.). The excess of Cl₂SO was eliminated by vacuum evaporation successively co-evaporating with anhydrous toluene. The crude obtained was derived chlorine 11 that was dissolved in anhydrous CH₂Cl₂ (15 mL) was cooled in a bath of ice water and 4 (343 mg, 1.1 mmol) and Et3N (0.145 mL) were added to it. The reaction mixture was allowed to reach room temperature and then the solvent was eliminated by vacuum evaporation. The crude obtained was purified by column chromatography (CH₂Cl₂-MeOH 20:1), obtaining 12 as a syrup (234 mg, 42%).

Compound 13: A solution of biotin 10 (120 mg, 1 mmol) in Cl₂SO (5 mL) was kept at room temperature (1 h.). The excess of Cl₂SO was eliminated by vacuum evaporation successively co-evaporating with anhydrous toluene. The crude obtained was the derived chlorine 11 that was dissolved in anhydrous THF (15 mL), and 8 (145 mg, 1.2 mmol) and Et3N (0.085 mL) dissolved in anhydrous THF (5 mL) were added to it. The reaction mixture was kept at room temperature during 10 min. then the solvent was eliminated by vacuum evaporation. The crude obtained was purified by column chromatography (AcOEt-MeOH 10:1 to 5:1), obtaining 13 as a syrup (140 mg, 63%).

Compound 14: A solution of biotin 10 (200 mg, 0.82 mmol) in Cl₂SO (5 mL) was kept at room temperature (1 h.). The excess of Cl₂SO was eliminated by vacuum evaporation successively co-evaporating with anhydrous toluene. The crude obtained was the derived chlorine 11 which was dissolved in anhydrous THF (15 mL), it was cooled in a bath of ice water and 9 (353 mg, 1 mmol) and Et3N (0.230 mL, 1.6 mmol) dissolved in anhydrous THF (5 mL) were added to it. The reaction mixture was allowed to reach room temperature and then the solvent was eliminated by vacuum evaporation. The crude obtained was purified by column chromatography (AcOEt-MeOH 5:1.) obtaining 14 as a syrup (438 mg, 92%).

### Example 3

### Synthesis of Single-Labelling Agents Based on Vinyl Sulphone Containing Fluorophores: Compounds 17, 18, 19 and 20

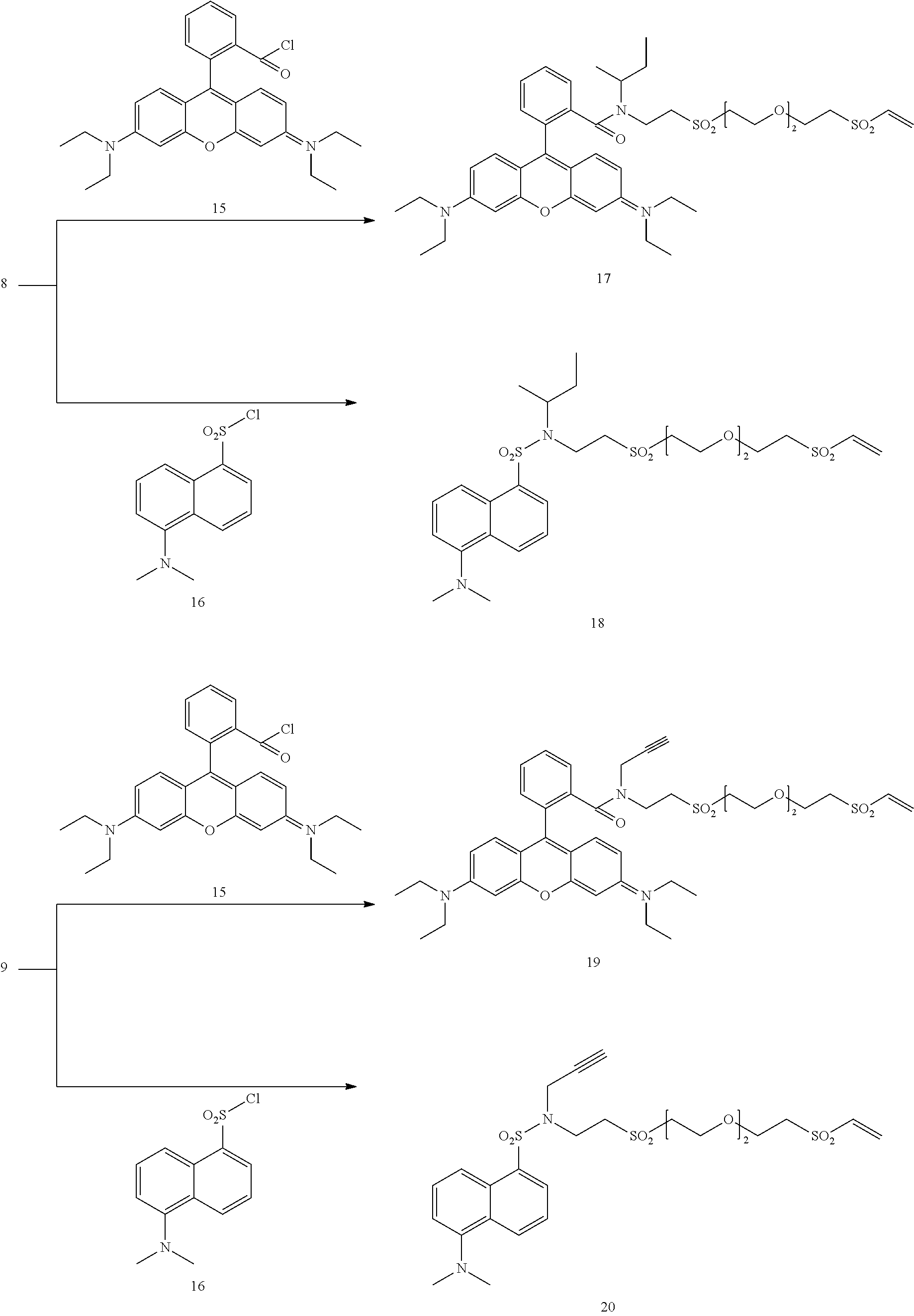

Compound 17: A solution of rhodamine B (100 mg, 0.2 mmol) in Cl₂SO (5 mL) was kept at room temperature (1 day). The excess of Cl₂SO was eliminated by vacuum evaporation successively co-evaporating with anhydrous toluene. The crude obtained was derived chlorine 15 which was dissolved in anhydrous THF (15 mL), 8 (64 mg, 0.17 mmol), and Et₃N (0.050 mL) dissolved in anhydrous THF (5 mL) were added to it. The reaction mixture was kept at room temperature during 10 min. then the solvent was eliminated by vacuum evaporation. The crude obtained was purified by column chromatography (CH₂Cl₂-MeOH 30:1 to 10:1) obtaining 17 as a syrup (64 mg, 44%).

Compound 18: 8 (150 mg, 0.40 mmol) and Et₃N (0.115 mL) were added to a solution of dansyl chloride 16 (130 mg, 0.48 mmol) in anhydrous acetonitrile (15 mL). The reaction mixture was kept at room temperature (2 days) then the solvent was eliminated by vacuum evaporation. The crude obtained was purified by column chromatography (AcOEt-hexane 1:1 to 3:1) obtaining 18 as a syrup (182 mg, 74%).

Compound 19: A solution of rhodamine B (195 mg, 0.41 mmol) in POCl₃ (5 mL) and 1,2-dichloroethane (5 mL) was subjected to reflux (16 h.). The excess of POCl₃ and the solvent were eliminated by vacuum evaporation successively co-evaporating with anhydrous toluene. The crude obtained contained the rhodamine chloride 15 that was directly used by solution in anhydrous THF (15 mL). It was cooled in a bath of ice water and 9 (174 mg, 0.49 mmol) and Et₃N (0.174 mL, 1.22 mmol) dissolved in anhydrous THF (5 mL) were added to it. The reaction mixture was allowed to reach room temperature and then the solvent was eliminated by vacuum evaporation. The crude obtained was purified by column chromatography (Cl₂CH₂-MeOH 20:1), obtaining 19 as a solid (272 mg, 86%).

Compound 20: 9 (180 mg, 0.50 mmol) and Et₃N (0.150 mL) were added to a solution of dansyl chloride 16 (275 mg, 1.0 mmol) in anhydrous acetonitrile (15 mL). The reaction mixture was kept at room temperature (3 days) then the solvent was eliminated by vacuum evaporation. The crude obtained was purified by column chromatography (AcOEt-hexane 1:1 to 3:1), obtaining 20 as a syrup (252 mg, 84%).

### Example 4

### Single Labelling of Proteins with Biotin Labelling Agents

### Example 4.1

### Labelling of the Bovine Serum Albumin (BSA) with Compound 13

The commercial Bovine Serum Albumin (BSA) (SIGMA A4503) (0.15 mM in water) was incubated with compound 13 (25.1 mM in 1:1 DMSO:water) with a stoichiometry 1:5 and 1:10 during 3 hours, after which the excess of product 13 was eliminated by dialysis. In order to evaluate if the BSA marked with biotin is recognized by avidin, it was incubated with fluorescent avidin (example 5.1) according to avidin stoichiometry:BSA 4:1 and 1:1 during 30 minutes and was analyzed by SDS-PAGE with mild denaturation (2 minutes at 100° C.). The fluorescence was visualized with a commercial transilluminator (λ=365 nm) and the protein was detected by Coomassie (FIG. 5). The result shows that the fluorescent avidin of example 5.1 recognizes the BSA marked with biotin and form stable complexes of high molecular weight which do not enter in the separating gel (14% acrylamide).

### Example 4.2

### Labelling of Green Fluorescent Protein (GFP) with Compound 12

GFP protein was obtained from an E. coli strain which was transformed with the pGFPCR plasmid that codifies for the UV variant of the GFP. Once the bacteria were subjected to lysis, the protein was purified using an IMAC column. The purified protein (2 mg/ml) was dialyzed before a PBS and was incubated with a 20 time excess of biotynation reactive 12 (considering that the protein GFP has a molecular weight of 27000). The incubation was kept at 4° C. during 12 h. and the reagent excess was blocked by ethanolamine addition. This sample is subsequently dialyzed before a PBS buffer. The obtained sample was directly used in an affinity chromatography on a biotin-silica column (according to the Spanish patent application: P200701850) saturated with avidin using a microfilter system with only 100 mg of the functionalized silica. The elution was carried out with HCl 0.2N and the eluate, after being lyophilized has been analyzed by MALDI-TOF spectrometry which shows molecular weight values 142965, 1 (avidin monomer) and 28565 (a molecule of GFP modified with 4 biotins).

### Example 5

### Single Labelling of Proteins with Fluorophore Labelling Agents

### Example 5.1

### Labelling of Avidin with Compound 18

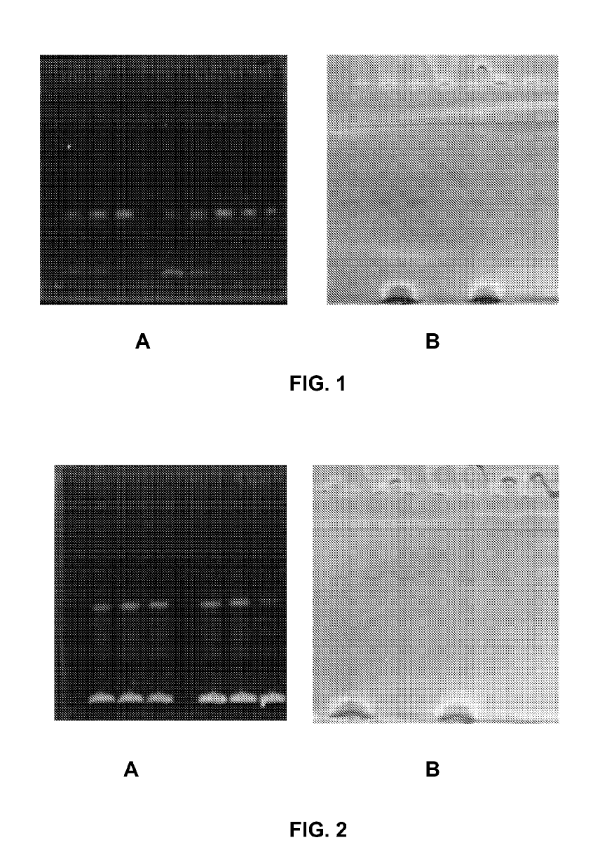

The commercial avidin (SIGMA A9275) (0.35 mM in water) was incubated with compound 18 (24.8 mM in 1:1 DMSO:water) with a stoichiometry 1:4 and 1:8 during 3.8 and 24 hours in HEPES 50 mM pH 8 and the result was analyzed in SDS-PAGE. The fluorescence was visualized with a commercial transilluminator (A=365 nm) and the protein was detected by Coomassie (FIG. 1). The optimal marking time was of the order of 8 hours, though with a 3-hour reaction it is already possible to detect the fluorescence. The marking is compatible with the detection by Coomassie and does not alter the capacity of the marked avidin of interacting with biotin.

### Example 5.2

### Labelling of Concanavalin A with Compound 17

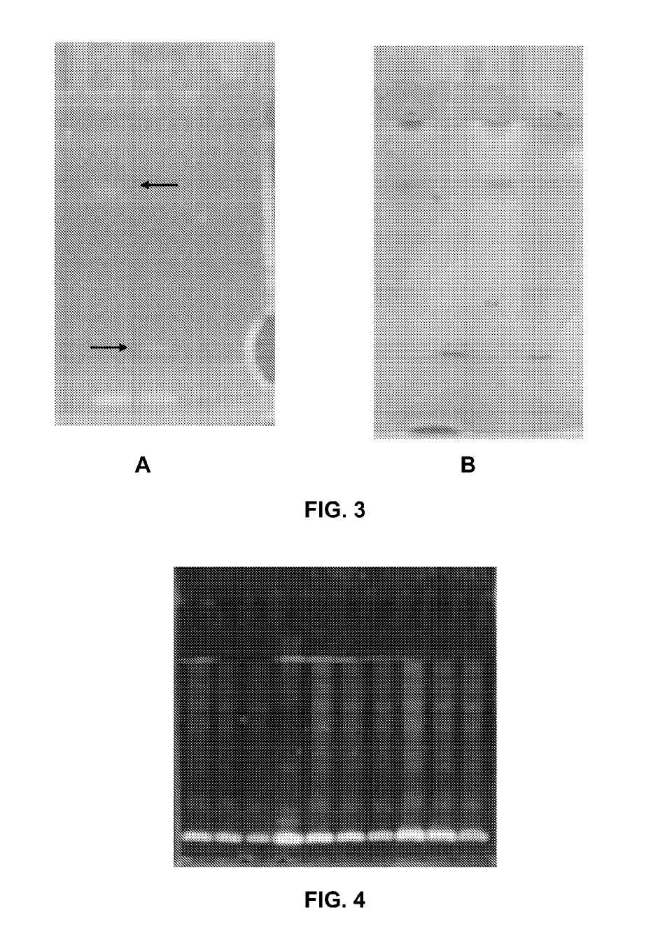

The commercial concanavalin A (SIGMA L7647) (0.39 mM in water) was incubated with compound 17 (18 mM in 1:1 DMSO:water) with a stoichiometry 1:5 and 1:8 during 3.8 and 24 hours in HEPES 50 mM pH 8 and the result was analyzed in SDS-PAGE (FIG. 2). The fluorescence was visualized with a commercial transilluminator (λ=365 nm) and the protein was detected by Coomassie. The fluorescence was so intense that no differences in relation to time were appreciated. High stoichiometries and reaction times favored the precipitation of the sample. The marking was compatible with the detection by Coomassie.

### Example 5.3

### Labelling Previous to Electrophoresis of BSA and Lysozyme with Rhodamine as an Alternative to Coomassie or Silver Staining of Electrophoresis Gels

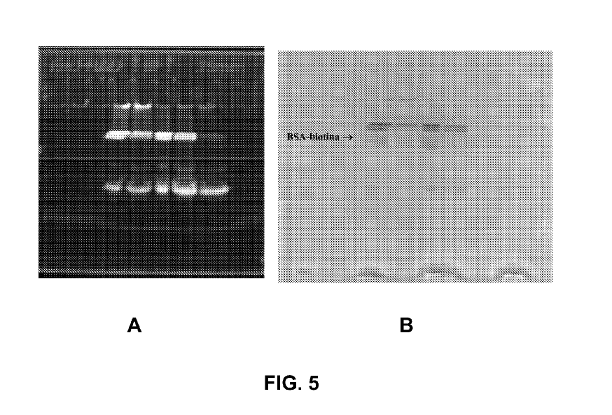

The fluorescent marking viability previous to electrophoresis was evaluated by the reaction during 10 minutes at 100° C. of 33 micrograms of the commercial Bovine Serum Albumin model proteins (SIGMA A4503) and lysozyme from egg with 3 micrograms of compound 17 in HEPES buffer 120 mM pH 8.8. Then 100 microlitres of load buffer (Tris-HCl 65.8 mM pH 6.8, glycerol 26% (v/v), SDS 2.1% (v/v), bromophenol blue 0.01% (w/v)) were added. The result was analyzed in SDS-PAGE (FIG. 3). The fluorescence was visualized with a commercial transilluminator (λ=365 nm) and the protein was detected by silver staining. The marking was compatible with the subsequent detection by silver staining and does not alter the migration pattern of any of the two proteins. The detection limit “de visu” is of the order, of 125 ng for both proteins.

### Example 5.4

### Labelling of Crude Pea Extract with Rhodamine as an Alternative to Coomassie or Silver Staining of Electrophoresis Gels

52 micrograms of pea extract were marked with 3, 6 and 9 micrograms of compound 17 by incubation during 10 minutes at 100° C. in HEPES 331 mM pH 8.8. Then 30 microlitres of load buffer were added and an electrophoresis was carried out (SDS-PGE) (FIG. 4). The result was typical of a crude extract, confirming the universality of marking, the viability as a system for fluorescent marking previous to electrophoresis and the compatibility with the subsequent Coomassie and/or silver staining.

1. Compound of general formula (I): 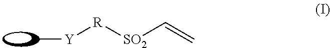
where:
Y is oxygen (O) or the group —N(R¹)CH₂CH₂SO₂; where;
R¹ is a radical, substituted or non-substituted, which is selected among an alkyl group (C₁-C₁₀) or a group (CH₂)ₘ C≡CH; where
m takes values from 1 to 10;
R is a radical, substituted or non-substituted, which is selected from the group comprising: —R²OCH₂CH₂, CH₂CH₂OR²OCH₂CH₂ or (CH₂CH₂O)ₙCH₂CH₂;
where
R² is a radical, substituted or non-substituted, that is selected from among an alkyl group (C₁-C₁₀) or a dialkylaryl group; where
n takes values from 2 to 20; and 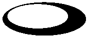
represents a labelling molecule.

2. Compound according to claim 1, where the labelling molecule is biotin or a fluorophore.

3. Compound according to claim 2, where the fluorophore is dansyl or rhodamine.

4. Compound according to claim 1, where R is —(CH₂CH₂O)ₙCH₂CH₂.

5. Compound according to claim 4, where n takes values from 1 to 3.

6. Compound according to claim 1, where Y is oxygen (O).

7. Compound according to claim 1, where Y is the group —N(R¹)CH₂CH₂SO₂ and R¹ is defined in claim 1.

8. Compound according to claim 7, where R¹ is an alkyl (C₂-C₆).

9. Compound according to claim 8, where R¹ is sec-butyl.

10. Compound according to claim 7, where R¹ is group (CH₂)ₘC≡CH and m takes values from 1 to 3.

11. Compound according to claim 10, where m is 1.

12. Compound according to claim 1, with formula:

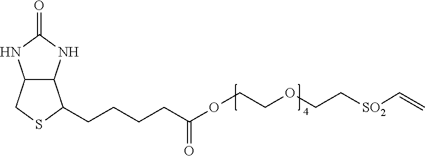

13. Compound according to claim 1, with formula:

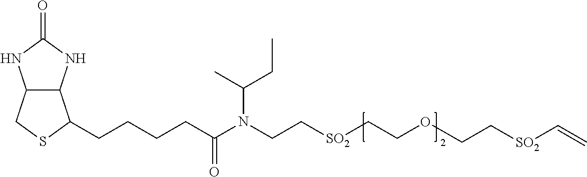

14. Compound according to claim 1, with formula:

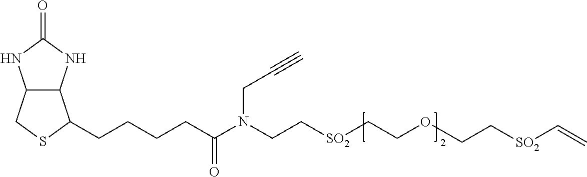

15. Compound according to claim 1, with formula:

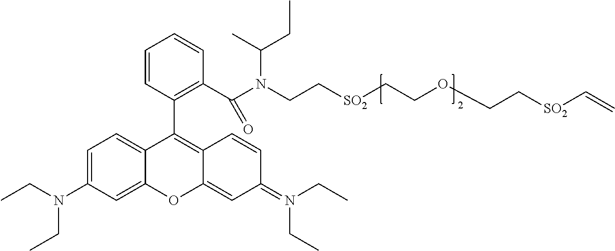

16. Compound according to claim 1, with formula:

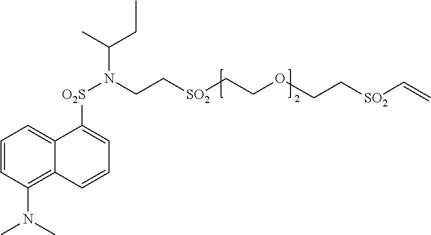

17. Compound according to claim 1, with formula:

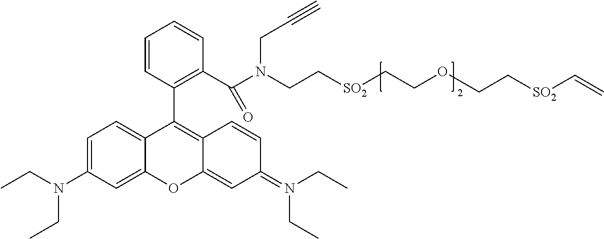

18. Compound according to claim 1, with formula:

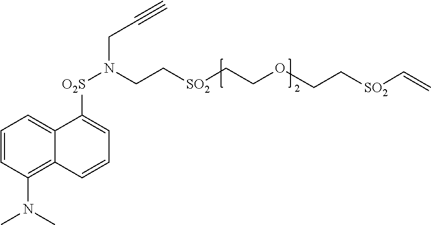

19. Method for obtaining a compound of general formula (I) according to claim 1, comprising the reaction of:
a. the compound of general formula (II): 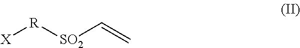
where: R is defined in claim 1; and
X is OH or the group —SO₂CH₂CH₂NH(R¹); where R¹ is
defined in claim 1.
b. with a labelling molecule or any derivatives thereof.

20. Method according to claim 19, where the derivatives of the labelling molecules are acid chlorides or sulphonyl chlorides.

21. Method according to claim 19, where the functionalized vinyl sulphone of step (a) is selected from among the compounds of formula:

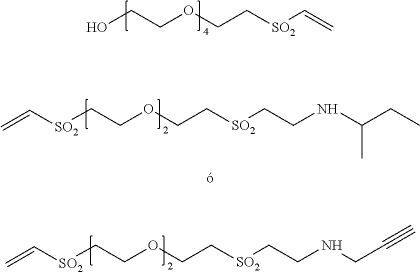

22. Use of a compound according to claim 1, as a labelling agent.

23. Labelling agent comprising a compound according to claim 1.

24. Use of the labelling agent according to claim 23, for biomolecule marking.

25. Use of the labelling agent according to claim 24, where the biomolecules are proteins.
